SEQUENCE LISTING (8 sequences)

SEQ ID NO: 1 - protein, 8 aa, Artificial Sequence
  Heavy Chain CDR1
GYSFTAYW

SEQ ID NO: 2 - protein, 8 aa, Artificial Sequence
  Heavy Chain CDR2
ILPGSDST

SEQ ID NO: 3 - protein, 11 aa, Artificial Sequence
  Heavy Chain CDR3
ARSGYYGSSQY

SEQ ID NO: 4 - protein, 10 aa, Artificial Sequence
  ESVDSYGNSF
ESVDSYGNSF

SEQ ID NO: 5 - protein, 3 aa, Artificial Sequence
  Light Chain CDR2
LTS

SEQ ID NO: 6 - protein, 9 aa, Artificial Sequence
  Light Chain CDR3
QQNAEDPRT

SEQ ID NO: 7 - protein, 137 aa, Artificial Sequence
  Heavy Chain Variable Domain
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGYYGSSQYWGQGTLVTVSS

SEQ ID NO: 8 - protein, 131 aa, Artificial Sequence
  Light Chain Variable Domain
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIK

(19) United States
(12) Patent Application Publication
(10) Pub. No.: US 2021/0148894 A1
(43) Pub. Date: May 20, 2021

(54) METHOD OF MONITORING RESPONSE TO A TREATMENT

(71) Applicant: Bionomics Limited, Thebarton (AU)

(72) Inventors: Tina LAVRANOS, Thebarton (AU); Kristen GEORGIOU, Thebarton (AU)

(21) Appl. No.: 17/046,719

(22) PCT Filed: Apr. 12, 2019
(86) PCT No.: PCT/AU2019/050334

§ 371 Date: Oct. 9, 2020

(30) Foreign Application Priority Data
Apr. 13, 2018 (AU) 2018901242

(51) Int. Cl. G01N 33/50; G01N 33/574

(52) U.S. Cl. CPC G01N 33/5041; G01N 33/57488; G01N 2333/8146; G01N 2800/52; G01N 2333/96494

(57) ABSTRACT

The present invention relates to the field of pharmacogenomics. In particular, provided herein is the use of pharmacodynamics markers in a method of monitoring the response of a subject to cancer treatment, wherein the cancer treatment comprises a Wnt/β-catenin signalling modulator (preferably an allosteric modulator of LGR5 receptor) and the monitoring comprises determining the ratio of Matrix Metalloproteinase-9 (MMP-9) and Tissue Inhibitor of Metalloproteinases 1 (TIMP-1) expression levels in a sample obtained from the subject.

### FIELD

The present invention relates to the field of pharmacogenomics. In particular, provided herein is the use of pharmacodynamics markers in a method of monitoring the response of a subject to cancer treatment.

### BACKGROUND

Colorectal cancer is the fourth most common type of cancer diagnosed in the United States with about 95000 cases in United States alone in 2017. It is the third leading cause of cancer-related deaths in women and the second leading cause in men in the United States. Colorectal cancer can start in the colon or rectum. Often the condition begins as a polyp which may form on the inner wall of the colon or rectum. Some of the polyps develop into cancers over time.

The leucine-rich repeat-containing G protein-coupled receptor 5 (LGR5) is a seven-transmembrane protein found on the surface of actively cycling intestinal stem cells and is an agonist of the Wnt/β-catenin signalling pathway. These LGR5 positive stem cells have prolonged lifespans and constant turnover, resulting in a high chance of them acquiring cancer causing mutations. These mutations are passed on to the daughter cells which can lead to cancer. LGR5 is found to be highly expressed in stage IV mestastatic colorectal cancer patients and is a target for cancer therapy.

Pharmacodynamic (PD) markers can be used to monitor the response of those patients receiving a therapeutic drug. If the PD markers indicate that the patient is not responding appropriately to the treatment, then the dosage of the therapeutic drug can be increased, decreased or discontinued. As such, there is a need to develop PD markers associated with LGR5 targeted therapies. This approach would ensure that patients receive the most appropriate treatment. Identification of the appropriate PD markers would also aid in understanding the mechanism of action upon administration.

### SUMMARY OF THE INVENTION

The present invention discloses methods of monitoring the response of a subject to a treatment with a WNT/β-catenin signalling modulator.

In one aspect, there is provided a method of monitoring the response of a subject to treatment with a WNT/β-catenin signalling modulator, the method comprising:
a) administering a WNT/β-catenin signalling modulator to a subject; and
b) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling modulator to the subject;
   wherein a change in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator.

In another aspect, there is provided a method of treating a subject having a hyper-proliferative disease with a WNT/β-catenin signalling modulator, the method comprising:
a) administering a WNT/β-catenin signalling modulator to a subject;
b) determining the ratio of MMP-9 and TIMP-1 expression levels in a sample obtained from the subject following administration of the WNT/β-catenin signalling modulator to the subject; wherein a change in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator; and
c) treating the subject found to be responsive to the WNT/β-catenin signalling modulator. In one embodiment, the WNT/β-catenin signalling modulator is an LGR5 receptor agonist or antagonist.

### BRIEF DESCRIPTION OF THE FIGURES

FIG. 1 is a graphical representation showing the assessment of the MMP-9/TIMP-1 ratio in patients grouped by AB1 dose and analysed as a group per treatment time-point. A) Mean±SEM of patient plasma MMP-9/TIMP-1 ratio per time-point at the 2.5 mg/kg dose level. B) Mean±SEM of patient plasma MMP-9/TIMP-1 ratio per time-point at the 5 mg/kg dose level. C) Mean±SEM of patient plasma MMP-9/TIMP-1 ratio per timepoint at the 10 mg/kg dose level. D) Mean±SEM of patient plasma MMP-9/TIMP-1 ratio per timepoint at the 15 mg/kg dose level. Significance was achieved in the 15 mg/kg AB1-treated group when comparing Baseline to Day 22 and Baseline to Cycle 2 Day 1. Statistical analyses performed using One-way ANOVA and Tukey's multiple comparisons test whereby p≤0.05* and p≤0.01**.

FIG. 2 is a graphical representation of the assessment of the MMP-9/TIMP-1 ratio in KRAS wildtype vs KRAS mutant patients who received 15 mg/kg AB1. A) Plasma MMP-9/TIMP-1 ratio of KRAS wildtype group analysed over one cycle of treatment. B) Plasma MMP-9/TIMP-1 ratio of KRAS mutant group analysed over one cycle of treatment. Significance was achieved in the 15 mg/kg AB1-treated KRAS mutant group when comparing Baseline to Cycle 2 Day 1 only. Statistical analyses performed using One-way ANOVA and Tukey's multiple comparisons test whereby p≤0.05* and p<0.01**.

### DETAILED DESCRIPTION OF THE INVENTION

The present invention discloses methods for monitoring the response of a subject to treatment with a WNT/β-catenin signalling inhibitor.

Provided herein is a method of monitoring the response of a subject to treatment with a WNT/β-catenin signalling modulator, the method comprising:
a) administering a WNT/β-catenin signalling modulator to a subject; and
b) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling modulator to the subject,
   wherein a change in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator.

In a further aspect there is provided a method of treating a subject having a hyper-proliferative disease with a WNT/β-catenin signalling modulator, the method comprising:
a) administering a WNT/β-catenin signalling modulator to a subject;
b) determining the ratio of MMP-9 and TIMP-1 expression levels in a sample obtained from the subject following administration of the WNT/β-catenin signalling modulator to the subject, wherein a change in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator; and
c) treating the subject found to be responsive to the WNT/β-catenin signalling modulator.

In one embodiment, there is provided a method of monitoring the response of a subject to treatment with a WNT/β-catenin signalling inhibitor, the method comprising:
a) administering a WNT/β-catenin signalling inhibitor to a subject;
b) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling inhibitor to the subject; and wherein a change in the ratio of MMP-9 and TIMP-1 expression levels as compared to the ratio of MMP-9 and TIMP-1 expression levels prior to administration of the WNT/β-catenin signalling inhibitor indicates that the subject is responsive to the WNT/β-catenin signalling inhibitor.

In one embodiment, there is provided a method of treating a subject having a hyper-proliferative disease with a WNT/β-catenin signalling inhibitor, the method comprising:
a) administering a WNT/β-catenin signalling inhibitor to a subject;
b) determining the ratio of MMP-9 and TIMP-1 expression levels in a sample obtained from the subject following administration of the WNT/β-catenin signalling inhibitor to the subject;
wherein a change in the ratio of MMP-9 and TIMP-1 expression levels as compared to the ratio of MMP-9 and TIMP-1 expression levels prior to administration of the WNT/β-catenin signalling inhibitor indicates that the subject is responsive to the WNT/β-catenin signalling inhibitor; and

treating the subject found to be responsive to the WNT/β-catenin signalling inhibitor.

In one embodiment, there is provided a method of monitoring the response of a subject to treatment with a WNT/β-catenin signalling agonist, the method comprising:
a) administering a WNT/β-catenin signalling agonist to a subject;
b) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling agonist to the subject; and
   wherein a change in the ratio of MMP-9 and TIMP-1 expression levels as compared to the ratio of MMP-9 and TIMP-1 expression levels prior to administration of the WNT/β-catenin signalling agonist indicates that the subject is responsive to the WNT/β-catenin signalling agonist.

In one embodiment, there is provided a method of monitoring the response of a subject to treatment with an LGR5 receptor agonist, the method comprising:
a) administering an LGR5 receptor agonist to a subject;
b) determining the ratio of MMP-9 and TIMP-1 expression levels in a sample obtained from the subject following administration of the LGR5 receptor agonist to the subject; and
   wherein a change in the ratio of MMP-9 and TIMP-1 expression levels as compared to the ratio of MMP-9 and TIMP-1 expression levels prior to administration of the LGR5 receptor agonist indicates that the subject is responsive to the LGR5 receptor agonist.

In one embodiment, there is provided a method of treating a subject having a hyper-proliferative disease with an LGR5 receptor agonist, the method comprising:
a) administering an LGR5 receptor agonist to a subject;
b) determining the ratio of MMP-9 and TIMP-1 expression levels in a sample obtained from the subject following administration of the LGR5 receptor agonist to the subject;
   wherein a change in the ratio of MMP-9 and TIMP-1 expression levels as compared to the ratio of MMP-9 and TIMP-1 expression levels prior to administration of the LGR5 receptor agonist indicates that the subject is responsive to the LGR5 receptor agonist; and
c) treating the subject found to be responsive to the LGR5 receptor agonist.

In one embodiment, there is provided a method of monitoring the response of a subject to treatment with an AB1 antibody, the method comprising:
c) administering an AB1 antibody to a subject;
d) determining the ratio of MMP-9 and TIMP-1 expression levels in a sample obtained from the subject following administration of the AB1 antibody to the subject; and
   wherein a decrease in the ratio of MMP-9 and TIMP-1 expression levels as compared to the ratio of MMP-9 and TIMP-1 expression levels prior to administration of the AB1 antibody indicates that the subject is responsive to the AB 1 antibody.

In one embodiment, there is provided a method of treating a subject having a hyper-proliferative disease with an AB1 antibody, the method comprising:
a) administering an AB1 antibody to a subject;
b) determining the ratio of MMP-9 and TIMP-1 expression levels in a sample obtained from the subject following administration of the AB1 antibody to the subject;
wherein a decrease in the ratio of MMP-9 and TIMP-1 expression levels as compared to the ratio of MMP-9 and TIMP-1 expression levels prior to administration of the AB1 antibody indicates that the subject is responsive to the AB1 antibody; and
c) treating the subject found to be responsive to the AB1 antibody.

In one embodiment, there is provided a method of monitoring the response of a subject to treatment with a WNT/β-catenin signalling modulator, the method comprising:
c) administering a WNT/β-catenin signalling modulator to a subject; and
d) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling modulator to the subject,
   wherein a change in the ratio of MMP-9 and TIMP-1 expression levels indicates that the subject is responsive to the WNT/β-catenin signalling modulator.

In one embodiment, there is provided a method of treating a subject having a hyper-proliferative disease with a WNT/β-catenin signalling modulator, the method comprising:

a) administering a WNT/β-catenin signalling modulator to a subject;
b) determining the ratio of MMP-9 and TIMP-1 expression levels in a sample obtained from the subject following administration of the WNT/β-catenin signalling modulator to the subject, wherein a change in the ratio of MMP-9 and TIMP-1 expression levels indicates that the subject is responsive to the WNT/β-catenin signalling modulator; and
e) treating the subject found to be responsive to the WNT/β-catenin signalling modulator.

In one embodiment, the subject is suffering from a hyper-proliferative disease. The hyper-proliferative disease may be a cancer.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a sub-stage of a Stage II cancer. One skilled in the art will appreciate that the classification of a Stage II cancer as either an early stage cancer or a late stage cancer depends on the particular type of cancer.

In one embodiment, the cancer is a cancer in an organ that harbours an LGR5+ cell. In one embodiment, the cancer is a cancer in an organ that harbours an LGR5+ stem cell. In one embodiment, the cancer is a metastatic cancer from an organ that harbours an LGR5+ stem cell.

In an embodiment the cancer is a cancer known to over-express LGR5 such as hepatocellular cancer (liver cancer) and gastric cancer (stomach cancer) and therefore have the potential to be treated by an LGR5 targeting antibody.

In one embodiment, the cancer is a cancer including colon cancer, colorectal cancer, pancreatic cancer, breast cancer, liver cancer, gastric cancer or lung cancer.

In some embodiments, the cancer is a cancer including colon cancer, metastatic colorectal cancer, metastatic pancreatic cancer, triple-negative breast cancer, or small cell lung cancer.

In some embodiments, the cancer is a cancer including colon cancer, colorectal cancer, pancreatic cancer, breast cancer, or lung cancer. In some embodiments, the cancer is a cancer including a colon cancer comprising an APC mutation, colon cancer comprising an KRAS mutation, metastatic colorectal cancer, metastatic pancreatic cancer, triple-negative breast cancer, or small cell lung cancer.

In one embodiment, the cancer is colorectal cancer. The term “colorectal cancer” can refer to colon cancer or rectal cancer. In another embodiment, the cancer is a pancreatic or gastric cancer.

The term “administering” refers to contacting, applying or providing a composition of the present invention to a subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

In one embodiment, the WNT/β-catenin signalling modulator is an allosteric modulator of LGR5 receptor. In one embodiment, the WNT/β-catenin signalling modulator is an LGR5 receptor inhibitor (or antagonist). In one embodiment, the WNT/β-catenin signalling inhibitor is an LGR5 receptor antagonist. The LGR5 receptor inhibitor or antagonist may lead to a decrease in WNT/β-catenin signalling downstream signalling.

In one embodiment, the WNT/β-catenin signalling modulator is an LGR5 receptor agonist. The LGR5 receptor agonist may bind to the LGR5 receptor and lead to an increase in WNT/β-catenin signalling downstream signalling through the LGR5 receptor. In one embodiment, the LGR5 receptor agonist is an R-Spondin ligand. In one embodiment, the LGR5 receptor agonist is an anti-LGR5 antibody or a fragment thereof. In one embodiment, the LGR5 receptor agonist is AB1 antibody or a fragment thereof.

In one embodiment, the method comprises administering AB1 at a dose of 15 mg/kg to a subject.

As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv), Fab fragments, F(ab′) fragments, disulfide-linked Fvs (sdFv) (including bi-specific sdFvs), and anti-idiotypic (anti-Id) antibodies, and epitope-binding fragments of any of the above. The antibodies of several embodiments provided herein may be monospecific, bispecific, trispecific or of greater multispecificity. Multispecific antibodies may be specific for different epitopes of a polypeptide or may be specific for both a polypeptide as well as for a heterologous epitope, such as a heterologous polypeptide or solid support material.

In one embodiment, the antibody is an antibody fragment (such as an scFV, ds-Fv, ds-ScFv, sd Ab and diabodies).

As used herein, LGR5 includes, but is not limited to, human LGR5 including the polypeptide of NCBI Accession No. NP 003658.1, or fragments thereof, which is encoded by the coding nucleotide sequence within NM 003667.2, or fragments thereof. The amino acid sequence and entire entry of NCBI Accession No. NP 003658.1 and nucleotide sequence and entire entry of NM 003667.2 are fully incorporated by reference in their entireties. Examples of LGR5 fragments contemplated herein include the LGR5 ectodomain, transmembrane domain, or intracellular domain and portions thereof.

Several embodiments relate to a hybridoma that produces the light chain and/or the heavy chain of an anti-LGR5 antibody, including the anti-LGR5 antibodies designated as 18G7H6A3 and 18G7H6A1, as described in detail in U.S. Pat. No. 9,546,214 B2, which is incorporated herein by reference in its entirety.

Various embodiments are directed to a vector comprising a nucleic acid molecule or molecules encoding a light chain and/or a heavy chain of an anti-LGR5 antibody, including any one of the anti-LGR5 antibodies designated as 18G7H6A3 and 18G7H6A1, as described in detail in U.S. Pat. No. 9,546,214 B2, which is incorporated herein by reference in its entirety.

In various embodiments, the glycosylation of the antibodies can be modified. For example, an aglycosylated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

In some embodiments, an anti-LGR5 antibody provided herein comprises a heavy chain CDR1 comprising GYSFTAYW (SEQ ID NO: 1), a heavy chain CDR2 comprising ILPGSDST (SEQ ID NO: 2), and a heavy chain CDR3 comprising ARSGYYGSSQY (SEQ ID NO: 3). In some embodiments, an anti-LGR5 antibody provided herein comprises a light chain CDR1 comprising ESVDSYGNSF (SEQ ID NO: 4), a light chain CDR2 comprising LTS (SEQ ID NO: 5), and a light chain CDR3 comprising QQNAEDPRT (SEQ ID NO: 6).

In one embodiment, the anti-LGR5 antibody (designated AB1) comprises a heavy chain CDR1 comprising GYSFTAYW (SEQ ID NO: 1), a heavy chain CDR2 comprising ILPGSDST (SEQ ID NO: 2), a heavy chain CDR3 comprising ARSGYYGSSQY (SEQ ID NO: 3), a light chain CDR1 comprising ESVDSYGNSF (SEQ ID NO: 4), a light chain CDR2 comprising LTS (SEQ ID NO: 5), and a light chain CDR3 comprising QQNAEDPRT (SEQ ID NO: 6).

In one embodiment, the anti-LGR5 antibody comprises a heavy chain variable domain having the sequence of:

(SEQ ID NO: 7)
MEWSWVFLFFLSVTTGVHSEVQLVQSGAEVKKPGESLRISCKGSGYSFT
AYWIEWVRQAPGKGLEWIGEILPGSDSTNYNEKFKGHVTISADKSISTA
YLQWSSLKASDTAVYYCARSGYYGSSQYWGQGTLVTVSS.

In one embodiment, the anti-LGR5 antibody comprises a light chain variable domain having the sequence of:

(SEQ ID NO: 8)
MSVPTQVLGLLLLWLTDARCDIVLTQSPASLAVSPGQRATITCRASESV
DSYGNSFMHWYQQKPGQPPKLLIYLTSNLESGVPDRFSGSGSGTDFTLT
INPVEANDAATYYCQQNAEDPRTFGGGTKLEIK.

Another embodiment includes the introduction of conservative amino acid substitutions in any portion of an anti-LGR5 antibody, such as AB1. It is well known in the art that “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine).

As used herein, the term “nucleic acid”, and equivalent terms such as polynucleotide, refers to a polymeric form of nucleotides of any length, such as ribonucleotides, deoxyribonucleotides or peptide nucleic acids (PNAs), that comprise purine and pyrimidine bases, or other natural, chemically or biochemically modified, non-natural, or derivatized nucleotide bases. The nucleic acid may be double stranded or single stranded. References to single stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof.

The WNT/β-catenin signalling modulator as decribed herein can be administered parenterally. The WNT/β-catenin signalling modulator may be administered directly into the blood stream, into tissue, into muscle, or into an internal organ. Administration may be systemic, e.g., to injection or infusion. Administration may be local. Suitable means for administration include intravenous, intraarterial, intrathecal, intraventricular, intraurethral, intrasternal, intracranial, subretinal, intravitreal, intra-anterior chamber, intramuscular, intrasynovial and subcutaneous. Suitable devices for administration include needle (including microneedle) injectors, needle-free injectors and infusion techniques.

The WNT/β-catenin signalling modulator will generally, but not necessarily, be administered as a formulation in association with one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a nonfunctional (e.g. processing aid or diluent) characteristic to the formulations. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

The term “sample” may be of any biological tissue or fluid. The sample may be a sample which is derived from a patient. Such samples include, but are not limited to, blood, serum, plasma, urine, saliva, semen, breast exudate, cerebrospinal fluid, tears, sputum, mucous, lymph, cytosols, ascites, pleural effusions, peritoneal fluid, amniotic fluid, bladder washes, and bronchioalveolar lavages, blood cells (e.g., white cells), tissue or biopsy samples (e.g., tumor biopsy), or cells therefrom. Biological samples may also include sections of tissues such as frozen sections taken for histological purposes.

In one embodiment, the method involves measuring the expression level of MMP-9 and TIMP-1 in a sample obtained from the subject. This may involve collecting a sample of biological material from the subject using methods that are known in the art. The step of measuring the expression levels of MMP-9 and TIMP-1 may be carried out by any suitable means, including ELISA, Western Blotting, RIA (radioimmunoassay), nucleic acid-based (e.g. polymerase chain reaction based techniques) or protein-based aptamer techniques, HPLC (high precision liquid chromatography), SPR (surface plasmon resonance), mass spectrometry. The ratio of the expression levels between MMP-9 and TIMP-1 (MMP-9/TIMP-1) may then be determined.

In one embodiment, the method comprises obtaining a sample from the subject prior to administering a WNT/β-catenin signalling modulator and determining the ratio of MMP-9 and TIMP-1 expression levels in the sample.

In another embodiment, the method comprises obtaining a sample from the subject following administrating a WNT/β-catenin signalling modulator and determining the ratio of MMP-9 and TIMP-1 expression levels in the sample.

In one embodiment, the method comprises comparing the ratio of MMP-9 and TIMP-1 expression levels in the sample obtained following administration of the WNT/β-catenin signalling modulator to the subject and in the sample obtained prior to administration of the WNT/β-catenin signalling modulator to the subject.

One or more additional biomarkers in addition to MMP-9 and TIMP-1 may be measured.

In one embodiment, the change in the ratio of MMP-9 and TIMP-1 expression levels is an increase in the ratio of MMP-9 and TIMP-1 expression levels.

In one embodiment, the increase in the ratio of MMP-9 and TIMP-1 expression levels indicates that the subject is responsive to the WNT/β-catenin signalling modulator. The increase in the ratio of MMP-9 and TIMP-1 may be 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 41 fold, 42 fold, 43 fold, 44 fold, 45 fold, 46 fold, 47 fold, 48 fold, 49 fold, 50 fold, 51 fold, 52 fold, 53 fold, 54 fold, 55 fold, 56 fold, 57 fold, 58 fold, 59 fold, 60 fold, 61 fold, 62 fold, 63 fold, 64 fold, 65 fold, 66 fold, 67 fold, 68 fold, 69 fold, 70 fold, 71 fold, 72 fold, 73 fold, 74 fold, 75 fold, 76 fold, 77 fold, 78 fold, 79 fold, 80 fold, 81 fold, 82 fold, 83 fold, 84 fold, 85 fold, 86 fold, 87 fold, 88 fold, 89 fold, 90 fold, 91 fold, 92 fold,93 fold, 94 fold, 95 fold, 96 fold, 97 fold, 98 fold, 99 fold or 100 fold, or an increase in between.

In one embodiment, the change in the ratio of MMP-9 and TIMP-1 expression levels is a decrease in the ratio of MMP-9 and TIMP-1 expression levels.

In one embodiment, the decrease in the ratio of MMP-9 and TIMP-1 expression levels indicates that the subject is responsive to the WNT/β-catenin signalling modulator. The decrease in the ratio of MMP-9 and TIMP-1 may be 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 31 fold, 32 fold, 33 fold, 34 fold, 35 fold, 36 fold, 37 fold, 38 fold, 39 fold, 40 fold, 41 fold, 42 fold, 43 fold, 44 fold, 45 fold, 46 fold, 47 fold, 48 fold, 49 fold, 50 fold, 51 fold, 52 fold, 53 fold, 54 fold, 55 fold, 56 fold, 57 fold, 58 fold, 59 fold, 60 fold, 61 fold, 62 fold, 63 fold, 64 fold, 65 fold, 66 fold, 67 fold, 68 fold, 69 fold, 70 fold, 71 fold, 72 fold, 73 fold, 74 fold, 75 fold, 76 fold, 77 fold, 78 fold, 79 fold, 80 fold, 81 fold, 82 fold, 83 fold, 84 fold, 85 fold, 86 fold, 87 fold, 88 fold, 89 fold, 90 fold, 91 fold, 92 fold,93 fold, 94 fold, 95 fold, 96 fold, 97 fold, 98 fold, 99 fold or 100 fold, or a decrease in between.

In one embodiment, the ratio of Matrix Metalloproteinase 9 (MMP-9) and Tissue Inhibitor of Metalloproteinases 1 (TIMP-1) expression levels is determined within a suitable time period following administration of the WNT/β-catenin signalling modulator to the subject. The time period may be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days or 90 days, or a period in between.

Provided herein are also methods of treating a subject. In one embodiment, the method further comprises treating the subject with the WNT/β-catenin signalling modulator, wherein the subject is found to be responsive to the WNT/β-catenin signalling modulator.

In one embodiment, there is provided a method for identifying a subset of subjects who are responsive to treatment to a WNT/β-catenin signalling modulator (e.g. an AB1 antibody). For example, it is shown that a particular subject of subjects with Kras mutation is particularly responsive to treatment with AB1 antibody based on the change in ratio of MMP-9 and TIMP-1 expression levels following administration of the WNT/β-catenin signalling modulator.

In one embodiment, there is provided a method of treating a subject having a hyper-proliferative disease, the method comprising administering a WNT/β-catenin signalling modulator (e.g. an AB1 antibody) to the subject, wherein the subject is identified to have a Kras mutation.

In one embodiment, there is provided a method of determining the therapeutically effective dose of a treatment with a WNT/β-catenin signalling modulator (e.g. an AB1 antibody). This may be determined by administering a particular dose of WNT/β-catenin signalling modulator to a subject and determining whether there is a change in ratio of MMP-9 and TIMP-1 expression levels following administration of the WNT/β-catenin signalling modulator.

Provided herein are compositions for determining the ratio of Matrix Metalloproteinase 9 (MMP-9) and Tissue Inhibitor of Metalloproteinases 1 (TIMP-1) expression levels in a sample. In one embodiment, there is provided a composition comprising an anti-MMP-9 antibody and a TIMP-1 antibody. The antibodies may be conjugated to one or more labels (e.g. flurorescent labels) to allow the detection of the MMP-9 and TIMP-1. In one embodiment, there is provided a composition comprising nucleic acid primers for amplifying and detecting MMP-9 and TIMP-1. The nucleic acid primers may be coupled or attached to one or more labels (e.g. fluorescent label) to allow for detection.

Provided herein are kits for determining the ratio of Matrix Metalloproteinase 9 (MMP-9) and Tissue Inhibitor of Metalloproteinases 1 (TIMP-1) expression levels in a sample. In one embodiment, the kit comprises one or more compartments comprising an anti-MMP-9 antibody or an anti-TIMP-1 antibody. The antibodies may be conjugated to one or more labels (e.g. flurorescent labels) to allow the detection of the MMP-9 and TIMP-1.

In one embodiment, there is provided the use of an anti-MMP-9 antibody and an anti-TIMP-1 antibody in the manufacture of a diagnostic assay. The diagnostic assay may be used for the monitoring of a response in a treatment following treatment with a WNT/β-catenin signalling modulator, wherein a change in the ratio of the expression levels of MMP-9 and TIMP-1 indicates that the subject is responsive to the WNT/β-catenin signalling modulator.

In one embodiment, there is provided the use of a WNT/β-catenin signalling modulator (e.g. an AB1 antibody) in the manufacture of a medicament for the treatment of hyper-proliferative disease in a subject. In one embodiment, the hyper-poliferative disease is cancer. In one embodiment, the cancer is a cancer with a Kras mutation.

As used in this application, the singular form “a,” “an,” and “the” include plural references unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

Materials and Methods

Patient Plasma Sample Collection

Patients from each site underwent blood collection at pre-dosing each week of the first cycle of AB1 treatment, followed by blood collection at pre-dosing on day one of each following cycle. Bloods were collected in Streck-BCT tubes and processed for plasma by spinning at 1900×g for 10 minutes, typically 24 hours following blood collection. Plasma samples were stored as 700 μl aliquots at −80° C. Control whole blood was collected from one healthy volunteer in a Streck-BCT tube and plasma was processed as per patient samples.

MMP-9 ELISA

MMP-9 ELISA plates were run as per manufacturer's instructions. Plasma MMP-9 protein concentrations were quantified using the generated standard curve slope. Standard curves generated for each ELISA plate are outlined in the appendices. Freshly thawed plasma was used for each plate. Healthy control, as well as an additional screen failure baseline sample were run on each ELISA plate as positive controls. Based on the manufacturer's instructions, a standard curve was generated on each ELISA plate when running patient plasmas diluted to the appropriate concentrations in recommended diluent. Based on initial optimisation plates, 1:300 dilution of plasma in diluent was used for MMP-9 detection.

TIMP-1 ELISA

TIMP-1 ELISA plates were run as per manufacturer's instructions. Plasma TIMP-1 protein concentrations were quantified using the generated standard curve slope. Standard curves generated for each ELISA plate are outlined in the appendices. Freshly thawed plasma was used for each plate. Healthy control, as well as an additional screen failure baseline sample from patient S03-006-1 were run on each ELISA plate as positive controls. Based on the manufacturer's instructions, a standard curve was generated on each ELISA plate when running patient plasmas diluted to the appropriate concentrations in recommended diluent. Based on initial optimisation plates, 1:100 plasma dilution was used for TIMP-1 detection.

MMP-9/TIMP-1 Ratio

Patient plasmas are processed to determine MMP-9 and TIMP-1 protein concentrations. The focus was initially to determine any potential imbalance in the MMP-9/TIMP-1 ratio over 1 cycle of treatment, analyzing baseline, day 15, day 22 and cycle 2 day 1 (pre-treatment) plasma samples. It was also of interest to determine whether patients who received an elongated treatment period exceeding cycle 2, demonstrated any sustained changes in the MMP-9/TIMP-1 ratio. As such, select patients from any cohort that were on study beyond cycle 2 were assessed, which included Patients 1, 2, 5, 9, 10 and 13. Once the plasma concentration of MMP-9 and TIMP-1 were determined on individual ELISA plates, the ratio of MMP-9/TIMP-1 was calculated per patient, per time-point (raw data can be found in referenced excel sheets). Each time a patient time-course was run, all samples were thawed fresh and analysed against the internal plate standard curve. All cohorts of patients (Cohort 1 2.5 mg/kg, Cohort 2 5 mg/kg, Cohort 3 10 mg/kg, Cohort 4 and Expansion Cohort 15 mg/kg) were statistically analysed using Graphpad Prism 7.02, whereby significance was achieved when p≤0.05 following an ANOVA and Tukey's multiple comparisons test.

### Example 1

Described herein is the analysis of AB1 patient plasma samples for the soluble proteins MMP-9 and TIMP-1 by ELISA. The MMP-9/TIMP-1 ratio was investigated herein to determine whether an imbalance in these two proteins exists, potentially indicative of changes in the tumor microenvironment. A statistically significant decrease in the ratio of plasma levels of MMP-9 and TIMP-1 was observed, which provides support for a pharmacodynamic effect following treatment with AB1 at 15 mg/kg, and in particular in patients that carry a KRAS mutation, which may be useful for patient selection in clinical investigations.

AB1 is a monoclonal antibody that binds the Wnt/P-catenin signaling agonist LGR5, found to be highly expressed in colorectal cancer (CRC) patients. The result of targeting LGR5 with a mAb in stage IV metastatic CRC patients is unknown, and while this Phase I study was conducted to demonstrate safety and tolerability, it also provided the opportunity to develop methodologies that may present useful information on patients treated with AB1 or highlight novel pharmacodynamic (PD) markers. While all patients on the AB1 study were stage IV metastatic CRC patients, they had significantly varied treatment histories, mutational profiles, genders, ages and metastatic sites. This extensive list of variables provided challenges in the capacity for data analyses.

Interestingly, preliminary gene expression analyses of patient tumor metastasis tissue from 2 patients treated with AB1 demonstrated changes in MMP-9 and/or TIMP-1 mRNA expression at day 22. It was asked whether AB1 influences the MMP-9/TIMP-1 ratio in patient plasma, which may be indicative of a change in the TME following treatment. MMP-9 and TIMP-1 are soluble proteins found at high concentrations in the plasma, easily detectable by ELISA. The MMP-9/TIMP-1 ratio was investigated to determine the potential imbalance in AB1 study patients and any influence of AB1 treatment on the tumor microenvironment.

### Example 2

Plasma MMP-9 and TIMP-1 Protein Concentration by ELISA

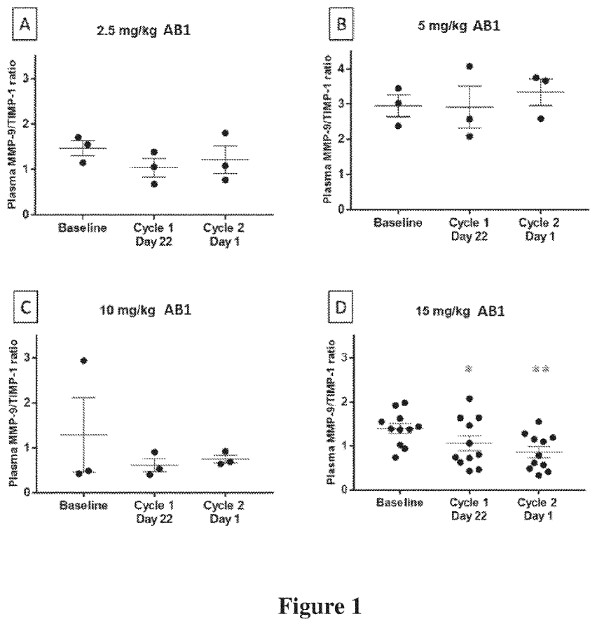

Plasma MMP-9 and TIMP-1 protein concentrations were expressed as a ratio in AB1-treated patient samples over one cycle of treatment. As outlined in FIG. 1, each patient may be individually traced over the first cycle of treatment, with key time-points graphed at baseline, day 22 and cycle 2 day 1 (pre-dose). While patients dosed with 2.5 mg/kg or 5 mg/kg (FIG. 1A) and 10 mg/kg (FIG. 1B) AB1 appear to have a steady MMP-9/TIMP-1 ratio over the first cycle of treatment, patients dosed with 15 mg/kg AB1 appear to have a trend towards a reduction in the MMP-9/TIMP-1 ratio at the cycle 2 day 1 time-point (FIG. 1C).

To determine whether there were any observable changes over a longer time-course of treatment, patients from a range of cohorts spanning 2.5-15 mg/kg AB1 dosing who remained on-study for 3 or more cycles were analysed for plasma MMP-9 and TIMP-1 protein concentration (FIG. 1D). The MMP-9/TIMP-1 ratio was calculated to determine any potential changes in the regulation of MMP-9 proteolytic activity within the tumor microenvironment in patients following AB1 treatment.

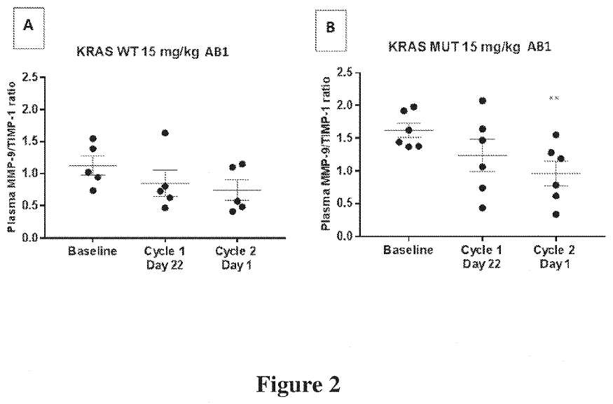

As the 15 mg/kg AB1 cohort demonstrated a reduction in the MMP-9/TIMP-1 ratio at cycle 2 day 1, this group of patients was further separated into KRAS wildtype and mutant (FIG. 2). The MMP-9/TIMP-1 ratio at the cycle 2, day 1 time-point as compared to baseline was significantly reduced in the KRAS mutant group (p<0.01), but not in the KRAS wildtype.

### CONCLUSIONS

Based on the data described herein, patients treated with 15 mg/kg AB1 when analysed as a group (mean±SEM) and not per patient, illustrate a reduction in the MMP-9/TIMP-1 ratio at both day 22 and cycle 2 day 1 following AB1 treatment. When this group is analysed further, patients who were carrying a KRAS mutation appear to be the drivers of the reduction in plasma MMP-9/TIMP-1 observed. Assessment of plasma MMP-9 and TIMP-1 protein concentration and analysis of the MMP-9/TIMP-1 ratio by ELISA has been demonstrated to be useful as a pharmacodynamic marker for AB1 treatment.

1. A method of monitoring the response of a subject to treatment with a WNT/β-catenin signalling modulator, the method comprising:
a) administering a WNT/β-catenin signalling modulator to a subject;
b) determining the ratio of Matrix Metalloproteinase 9 (MMP-9) and Tissue Inhibitor of Metalloproteinases 1 (TIMP-1) expression levels in a sample obtained from the subject following administration of the WNT/β-catenin signalling modulator to the subject; and
wherein a change in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator.

2. The method of claim 1, wherein the subject is suffering from a hyper-proliferative disease.

3. The method of claim 1, wherein the hyper-proliferative disease is cancer.

4. The method of claim 3, wherein the cancer is a cancer including colon cancer, colorectal cancer, pancreatic cancer, breast cancer, gastric cancer, liver cancer or lung cancer.

5. The method of claim 1, wherein the control sample is a sample obtained from the subject prior to administering the WNT/β-catenin signalling modulator.

6. The method of claim 1, wherein the WNT/β-catenin signalling modulator is an allosteric modulator of LGR5 receptor.

7. The method of claim 1, wherein the WNT/β-catenin signalling modulator is an AB1 antibody or fragment thereof.

8. The method of claim 1, wherein the method further comprises treating the subject with the WNT/β-catenin signalling modulator, wherein the subject is found to be responsive to the WNT/β-catenin signalling modulator.

9. A method of treating a subject having a hyper-proliferative disease with a WNT/β-catenin signalling modulator, the method comprising:
a) administering a WNT/β-catenin signalling modulator to a subject;
b) determining the ratio of MMP-9 and TIMP-1 expression levels in a sample obtained from the subject following administration of the WNT/β-catenin signalling modulator to the subject;
wherein an increase or decrease in the ratio of MMP-9 and TIMP-1 expression levels as compared to the ratio of MMP-9 and TIMP-1 expression levels prior to administration of the WNT/β-catenin signalling modulator indicates that the subject is responsive to the WNT/β-catenin signalling modulator; and
c) treating the subject found to be responsive to the WNT/β-catenin signalling modulator

10. The method of claim 9, wherein the hyper-proliferative disease is cancer.

11. The method of claim 10, wherein the cancer is a cancer including colon cancer, colorectal cancer, pancreatic cancer, breast cancer, gastric cancer, liver cancer or lung cancer.

12. The method of claim 9, wherein the control sample is a sample control sample is a sample obtained from the subject prior to administering the WNT/β-catenin signalling modulator.

13. The method of claim 9, wherein the WNT/β-catenin signalling modulator is an allosteric modulator of LGR5 receptor.

14. The method of claim 13, wherein the WNT/β-catenin signalling modulator is Ab1 antibody or fragment thereof.
